(19) United States
(12) Patent Application Publication
(10) Pub. No.: US 2016/0281142 A1
(43) Pub. Date: Sep. 29, 2016

(54) METHODS FOR PREDICTING OVERWEIGHT RISK FOR PETS AND ADULT PERCENT BODY FAT

(71) Applicant: NESTEC SA, Vevey (CH)

(72) Inventors: Gail Czarnecki-Maulden, Edwardsville, IL (US); Ziad S. Ramadan, Manchester, MO (US); Michael Yabes Manuzon, Frontenac, MO (US)

(21) Appl. No.: 15/076,866

(22) Filed: Mar. 22, 2016

Related U.S. Application Data
(60) Provisional application No. 62/138,100, filed on Mar. 25, 2015.

(51) Int. Cl. C12Q 1/68

(52) U.S. Cl. CPC C12Q 1/689; C12Q 2600/124

(57) ABSTRACT

The invention provides methods for determining overweight risk in a companion animal and to predict percent body fat in a young animal upon maturity. In one embodiment, a method for determining overweight risk in a companion animal can comprise measuring a relative abundance of bacteria from a microbiome of the companion animal; comparing the relative abundance of the bacteria to a relative abundance of the bacteria in a lean microbiome profile or in an overweight microbiome profile; and determining that the companion animal is at risk for being overweight if the relative abundance of bacteria is within the overweight microbiome profile or if the relative abundance of bacteria is outside the lean microbiome profile.

### CROSS REFERENCE TO RELATED APPLICATIONS

This application claims priority to U.S. Provisional Application No. 62/138,100 filed Mar. 25, 2015, the disclosure of which is incorporated herein by this reference.

### BACKGROUND OF THE INVENTION

1. Field of the Invention

This invention relates generally to the health of companion animals, and, more specifically to determinations of propensity of a companion animal to become overweight and predicted percent body fat of a companion animal upon maturity.

2. Description of Related Art

Many pet owners purchase pet foods at retail locations in consideration of their pets' life stage, body condition, activity level etc., but without the benefit of examination or advice by a pet expert such as a veterinarian or an animal nutritionist. Many pet owners, while making decisions to purchase appropriate food, incorrectly assess the body condition of their pet, even when shown a visual chart. The problem is more acute for owners of overweight pets, since it has been determined that only 1 out of 7 owners of overweight pets correctly recognize their pet as overweight. Since these pet owners do not recognize overweight conditions of their pets, they are therefore unable to choose an appropriate calorie pet food for their pet, and the health of the pet may be jeopardized as a result. Further, the pet may not be correctly diagnosed as over-weight until the assistance of an animal expert is requested.

Obesity is a major health concern for pets, both in dogs and cats. Approximately 30% of cats and dogs are overweight. Obesity leads to disease and shorter life span of the animal. Once a pet is overweight, it can be very difficult to decrease body weight of the pet and to prevent weight gain after weight loss.

While an animal expert, for example, a veterinarian or animal nutritionist, is more likely to determine with a higher degree of objectivity and probability the body condition score (BCS) of pets leading to more accurate diagnosis of obesity, such scoring systems still include a subjective element in the assessment process. Diagnosis is particularly difficult for pet that have an abundant hair coat. Additionally, many pet owners do not have their pets examined by an animal expert.

Methods for identifying obesity have included determination of body fat by DEXA (dual energy X-ray Absorptiometry) and total body water. These methods are not readily available to pet owners or animal experts.

As such, there remains a need for methods to assess overweight risk in pets.

### SUMMARY OF THE INVENTION

It is, therefore, an object of the present invention to provide methods useful for maintaining the health of a companion animal.

It is another object of the present invention to provide methods to predict an overweight risk for a companion animal.

It is still another object of the present invention to provide methods for predicting percent body fat upon maturity of a young companion animal.

In one embodiment, a method for determining overweight risk in a companion animal can comprise measuring a relative abundance of bacteria from a microbiome of the companion animal including at least two bacterium selected from the group consisting of Bifidobacterium longum, Coriobacteriaceae, [Eubacterium] cylindroides, Bifidobacterium adolescentis, Megasphaera, Bulleidia, Collinsella spp, Bifidobacteriumceae, Collinsella stercoris, Butyrivibrio, Bulleidia p_1630_c5, Dialister, Slackia spp, Prevotella copri, Catenibacterium, Megamonas, Lactobacillus ruminis, Clostridiaceae, Desulfovibrio, Clostridium, Streptococcus luteciae, Clostridium perfringens, Oscillospira, Clostridium hiranonis, Dorea spp, [Paraprevotellaceae] [Prevotella], Prevotella, Parabacteroides distasonis, Coprococcus spp, Sediminibacterium, Comamonadaceae, SMB53, Ruminococcus spp, S24_7_g, Bilophila, Parabacteroides, and Dorea formicigenerans; comparing the relative abundance of the bacteria to a relative abundance of the bacteria in a lean microbiome profile or in an overweight microbiome profile; and determining that the companion animal is at risk for being overweight if the relative abundance of bacteria is within the overweight microbiome profile or if the relative abundance of bacteria is outside the lean microbiome profile.

In another embodiment, a method of predicting percent of adult body fat for a companion animal having an age from 1 day to 6 months can comprise measuring the relative abundance of bacteria from a microbiome of the companion animal including Coprococcus spp, Candidatus Arthromitus spp, Turicibacter spp, [Eubacterium] biforme, Bifidobacterium spp, Streptococcus spp, Collinsella spp, Dorea spp, Clostridiales, Slackia spp, Erysipelotrichaceae, Faecalibacterium prausnitzii, Bacteroides spp, Ruminococcus spp, Phascolarctobacterium spp, Bacteroides plebeius; and calculating the percent of adult body fat according to the equation:

$$Predicted adult body fat \% = ( about ( - 30 ) \times ( relative abundance of Coprococcus spp ) ) + \\ ( about ( - 18.5 ) \times ( relative abundance of CandidatusArthromitus spp ) ) + \\ ( about ( - 1.5 ) \times ( relative abundance of Turicibacter spp ) ) + ( about ( - 0.1 ) \times ( relative abundance of [ Eubacterium ] biforme ) ) + ( about ( - 0.19 ) \times ( relative abundance of Bifidobacterium spp ) ) + ( about ( - 0.05 ) \times ( relative abundance of Streptococcus spp ) ) + ( about ( 0.10 ) \times ( relative abundance of Collinsella spp ) ) + ( about ( 0.4 ) \times ( relative abundance of Dorea spp ) ) + ( about ( 0.6 ) \times ( relative abundance of Clostridiales ) ) + ( about ( 3.4 ) \times ( relative abundance of Slackia spp ) ) + ( about ( 9 ) \times ( relative abundance of Erysipelotrichceae ) ) + ( about ( 11 ) \times ( relative abundance of Faecalibacterium prausnitzii ) ) + ( about ( 21 ) \times ( relative abundance of Bacteroides spp ) ) + ( about ( 24 ) \times ( relative abundance of Ruminococcus spp ) ) + ( about ( 26 ) \times ( relative abundance of Phascolarctobacterium spp ) ) + ( about ( 69 ) \times ( relative abundance of Bacteroides plebeius ) ) .$$

### DETAILED DESCRIPTION OF THE INVENTION

### Definitions

The term “companion animal” is any domesticated animal, and includes, without limitation, cats, dogs, rabbits, guinea pigs, ferrets, hamsters, mice, gerbils, horses, cows, goats, sheep, donkeys, pigs, and the like. In one example, the companion animal can be a dog or cat.

The term “lean microbiome profile” refers to bacteria of the microbiome including at least two of Clostridiaceae, Desulfovibrio, Clostridium, Streptococcus luteciae, Clostridium perfringens, Oscillospira, Clostridium hiranonis, Dorea spp, [Paraprevotellaceae] [Prevotella], Prevotella, Parabacteroides distasonis, Coprococcus spp, Sediminibacterium, Comamonadaceae, SMB53, Ruminococcus spp, S24_7_g, Bilophila, Parabacteroides, and Dorea formicigenerans, of a companion animal that is not overweight; i.e., that is within 15% its ideal adult body weight. In one embodiment, the lean microbiome profile can be for a cat.

The term “overweight microbiome profile” refers to bacteria of the microbiome including at least two of Bifidobacterium longum, Coriobacteriaceae, [Eubacterium] cylindroides, Bifidobacterium adolescentis, Megasphaera, Bulleidia, Collinsella spp, Bifidobacteriumceae, Collinsella stercoris, Butyrivibrio, Bulleidia p_1630_c5, Dialister, Slackia spp, Prevotella copri, Catenibacterium, Megamonas, and Lactobacillus ruminis, of a companion animal that is 15% over its ideal adult body weight. For example, for cats and dogs, ideal adult body weight can be determined by body condition scoring or other methods as identified in Table 1 of “The growing problem of obesity in dogs and cats? by German, A J, J Nutr. 1940s-1946s (2006)) or as discussed in Burkholder W J, Toll P W. Obesity. In: Hand M S, Thatcher C D, Reimillard R L, Roudebush P, Morris M L, Novotny B J, editors. Small animal clinical nutrition, 4th edition. Topeka, K S: Mark Morris Institute. 2000; p. 401-30. In one embodiment, the overweight microbiome profile can be for a cat.

The term “about” includes all values within a range of 5% of the stated number. In one embodiment, “about” includes all values within a range of 2%, and in one aspect, within 1%.

The term “individual” when referring to an animal means an individual animal of any species or kind.

The term “microbiome” refers to bacteria and other microorganisms found in the intestinal tract of a companion animal.

As used throughout, ranges are used herein in shorthand, so as to avoid having to set out at length and describe each and every value within the range. Any appropriate value within the range can be selected, where appropriate, as the upper value, lower value, or the terminus of the range.

As used herein, embodiments, aspects, and examples using “comprising” language or other open-ended language can be substituted with “consisting essentially of” and “consisting of” embodiments.

As used herein and in the appended claims, the singular form of a word includes the plural, and vice versa, unless the context clearly dictates otherwise. Thus, the references “a”, “an”, and “the” are generally inclusive of the plurals of the respective terms. For example, reference to “a kitten” or “a method” includes a plurality of such “kittens” or “methods”. Reference herein, for example to “a bacterium” includes a plurality of such bacteria, whereas reference to “pieces” includes a single piece. Similarly, the words “comprise”, “comprises”, and “comprising” are to be interpreted inclusively rather than exclusively. Likewise the terms “include”, “including” and “or” should all be construed to be inclusive, unless such a construction is clearly prohibited from the context. Where used herein the term “examples,” particularly when followed by a listing of terms is merely exemplary and illustrative, and should not be deemed to be exclusive or comprehensive.

The methods and compositions and other advances disclosed here are not limited to particular methodology, protocols, and reagents described herein because, as the skilled artisan will appreciate, they may vary. Further, the terminology used herein is for the purpose of describing particular embodiments only, and is not intended to, and does not, limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, certain compositions, methods, articles of manufacture, or other means or materials are described herein.

All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved. Full citations for publications not cited fully within the specification are set forth at the end of the specification.

### The Invention

The present inventors have discovered that overweight risk can be determined by measuring various levels of bacteria from gut microbiome of a companion animal and comparing to an overweight microbiome profile or a lean microbiome profile from comparative animals. Further, a predictive model for adult body fat has been developed for young companion animals. The present methods can use biomarkers spanning multiple genuses, families, orders, classes, and even phyla. Notably, the present inventors have discovered that the present biomarkers do not correspond to those found in humans. Specifically, the present inventors have discovered firmicutes that are typically correlated with being overweight in humans and other species (e.g., rodents) were not found to be dispostive as a phylum for cats. Particularly, some firmicutes predicted development of being overweight and others predicted remaining lean in the present study.

As such, in one embodiment, a method for determining overweight risk in a companion animal can comprise measuring a relative abundance of bacteria from a microbiome of the companion animal including at least two bacterium selected from the group consisting of Bifidobacterium longum, Coriobacteriaceae, [Eubacterium] cylindroides, Bifidobacterium adolescentis, Megasphaera, Bulleidia, Collinsella spp, Bifidobacteriumceae, Collinsella stercoris, Butyrivibrio, Bulleidia p_1630_c5, Dialister, Slackia spp, Prevotella copri, Catenibacterium, Megamonas, Lactobacillus ruminis, Clostridiaceae, Desulfovibrio, Clostridium, Streptococcus luteciae, Clostridium perfringens, Oscillospira, Clostridium hiranonis, Dorea spp, [Paraprevotellaceae] [Prevotella], Prevotella, Parabacteroides distasonis, Coprococcus spp, Sediminibacterium, Comamonadaceae, SMB53, Ruminococcus spp, S24_7 g, Bilophila, Parabacteroides, and Dorea formicigenerans; comparing the relative abundance of the bacteria to a relative abundance of the bacteria in a lean microbiome profile or in an overweight microbiome profile; and determining that the companion animal is at risk for being overweight if the relative abundance of bacteria is within the overweight microbiome profile or if the relative abundance of bacteria is outside the lean microbiome profile.

As discussed herein, the lean microbiome profile can include those bacteria found in a companion animal of the same breed, age, and/or gender that is healthy and of normal weight. In one embodiment, the present method can include comparing to the lean microbiome profile. Such a lean microbiome profile can include at least two bacterium selected from the group consisting of: Clostridiaceae, Desulfovibrio, Clostridium, Streptococcus luteciae, Clostridium perfringens, Oscillospira, Clostridium hiranonis, Dorea spp, [Paraprevotellaceae] [Prevotella], Prevotella, Parabacteroides distasonis, Coprococcus spp, Sediminibacterium, Comamonadaceae, SMB53, Ruminococcus spp, S24_7_g, Bilophila, Parabacteroides, and Dorea formicigenerans. In one aspect, the relative abundance of Clostridiaceae can range from 0.07% to 6.7%. In another aspect, the relative abundance of Desulfovibrio can range from 0.001% to 0.75%. In still another aspect, the relative abundance of Clostridium can range from 0.001% to 7.7%. In yet another aspect, the relative abundance of Streptococcus luteciae can range from 0.001% to 3%. In another aspect, the relative abundance of Clostridium perfringens can range from 0.001% to 1.1%. In another aspect, the relative abundance of Oscillospira can range from 0.02% to 0.77%. In another aspect, the relative abundance of Clostridium hiranonis can range from 0.9% to 17%. In another aspect, the relative abundance of Dorea spp can range from 0.001% to 1%. In another aspect, the relative abundance of [Paraprevotellaceae] [Prevotella] can range from 0.001% to 6.5%. In another aspect, the relative abundance of Prevotella can range from 0.001% to 0.6%. In another aspect, the relative abundance of Parabacteroides distasonis can range from 0.001 to 0.4%. In another aspect, the relative abundance of Coprococcus spp can range from 0.001% to 1.6%. In another aspect, the relative abundance of Sediminibacterium can range from 0.001% to 0.15%. In another aspect, the relative abundance of Comamonadaceae can range from 0.001% to 0.31%. In another aspect, the relative abundance of SMB53 can range from 0.03% to 0.8%. In another aspect, the relative abundance of Ruminococcus spp can range from 0.001% to 1.6%. In another aspect, the relative abundance of S24_7_g can range from 0.001% to 23%. In another aspect, the relative abundance of Bilophila can range from 0.001% to 0.1%. In another aspect, the relative abundance of Parabacteroides can range from 0.001% to 1.4%. In another aspect, the relative abundance of Dorea formicigenerans can range from 0.001% to 0.65%.

As discussed herein, the overweight microbiome profile can include those bacteria found in a companion animal of the same species, breed, age, and/or gender that is 15% more than the normal weight of the animal. In one embodiment, the present method can include comparing to the overweight microbiome profile. Such an overweight microbiome profile can include at least two bacterium selected from the group consisting of: Bifidobacterium longum, Coriobacteriaceae, [Eubacterium] cylindroides, Bifidobacterium adolescentis, Megasphaera, Bulleidia, Collinsella spp, Bifidobacteriumceae, Collinsella stercoris, Butyrivibrio, Bulleidia p_1630_c5, Dialister, Slackia spp, Prevotella copri, Catenibacterium, Megamonas, and Lactobacillus ruminis. In one aspect, the relative abundance of Bifidobacterium longum can range from 0.001% to 1.61%. In another aspect, the relative abundance of Coriobacteriaceae can range from 0.001% to 24.1%. In still another aspect, the relative abundance of [Eubacterium] cylindroides can range from 0.06% to 1%. In yet another aspect, the relative abundance of Bifidobacterium adolescentis can range from 0.001% to 17.3%. In another aspect, the relative abundance of Megasphaera can range from 0.001% to 12.5%. In another aspect, the relative abundance of Bulleidia can range from 0.001% to 3.4%. In another aspect, the relative abundance of Collinsella spp can range from 0.44% to 6.5%. In another aspect, the relative abundance of Bifidobacteriumceae can range from 0.065% to 0.95%. In another aspect, the relative abundance of Collinsella stercoris can range from 0.28% to 2%. In another aspect, the relative abundance of Butyrivibrio can range from 0.001% to 0.14%. In another aspect, the relative abundance of Bulleidia p_1630_c5 can range from 0.4 to 1.9%. In another aspect, the relative abundance of Dialister can range from 0.001% to 5.9%. In another aspect, the relative abundance of Slackia spp can range from 0.01% to 0.32%. In another aspect, the relative abundance of Prevotella copri can range from 2% to 18%. In another aspect, the relative abundance of Catenibacterium can range from 0.001% to 3.5%. In another aspect, the relative abundance of Megamonas can range from 0.001% to 0.19%. In another aspect, the relative abundance of Lactobacillus ruminis can range from 0.001% to 4.3%.

As discussed herein, the present method can include comparing bacteria from different genuses. In one aspect, the present method can include comparing bacteria from different families. In another aspect, the present method can include comparing bacteria from different orders. In yet another aspect, the present method can include comparing bacteria from different classes. In still another aspect, the present method can include comparing bacteria from different phyla. Additionally, while the present method generally includes the comparison of two bacterium; multiple bacteria can also be used. In one aspect, the bacteria can include at least 3 bacterium. In one specific aspect, the bacteria can include Megasphaera, Bifidobacterium, and Prevotella copri. In another aspect, the bacteria can include at least 4 bacterium. In still another aspect, the bacteria can include 5 bacterium. In other aspects, the bacteria can include 6, 7, 8, 9, 10, or more bacterium.

Generally, the bacteria are compared to a lean or overweight microbiome profile. Such comparison can include bacteria from different biological classifications, e.g. two different genuses or phyla, within a single profile. As such, an overweight risk assessment can include measuring multiple bacteria from different biological classifications and comparing the relative abundance of the bacteria to the relative abundance of bacteria within the overweight microbiome profile or the lean microbiome profile. Additionally, bacteria can be used belonging to a phylum, order, or class that has members in both the overweight microbiome profile and the lean microbiome profile, e.g., firmicutes.

The present bacteria referenced herein have been identified according to current known classification. Additionally, if the current classification is not known, the bacteria have been identified using the following operational taxonomic unit (OTU) numbers according to Tables 1 and 2:

TABLE 1

| Identification* | OTU numbers | |
|---|---|---|
| p_Bacteroidetes_c_Bacteroidia_{—} | 4376649 | 321811 |
| o_Bacteroidales_f_Bacteroidaceae_{—} | 4331736 | 3439403 |
| g_Bacteroides_s_{—} | 2189140 | 174978 |
| p_Firmicutes_c_Clostridia_{—} | 132784 | |
| o_Clostridiales_f_Veillonellaceae_{—} | | |
| g_Phascolarctobacterium_s_{—} | | |
| p_Firmicutes_c_Clostridia_{—} | 299837 | |
| o_Clostridiales_f_Ruminococcaceae_{—} | 4342682 | |
| g_Faecalibacterium_s_prausnitzii | 158438 | |
| p_Firmicutes_c_Erysipelotrichi_{—} | 3413566 | 1145262 |
| o_Erysipelotrichales_f_Erysipelotrichaceae_g_s_{—} | 4395065 | 592616 |
| | 4390365 | |

| p_Actinobacteria_c_Coriobacteriia_{—} | 367068 | | |
|---|---|---|---|
| o_Coriobacteriales_f_Coriobacteriaceae_{—} | 4339547 | | |
| g_Slackia_s_{—} | 347783 | | |
| p_Firmicutes_c_Clostridia_{—} | 337636 | 678717 | 321560 |
| o_Clostridiales_f_{—} | 295312 | 2657412 | 70137 |
| | 158540 | 4469233 | 988932 |
| | 181083 | 2500766 | 191945 |
| | 4306036 | 186057 | 303269 |
| | 175967 | 233881 | 146564 |
| | 184991 | 166099 | 322840 |
| | 4437746 | 196333 | 2575651 |
| | 621700 | 4417708 | |

| | | |
|---|---|---|
| p_Firmicutes_c_Clostridia_{—} | 259922 | 177403 |
| o_Clostridiales_f_Ruminococcaceae_{—} | 181035 | 4456702 |
| g_Ruminococcus_s_{—} | | |
| p_Bacteroidetes_c_Bacteroidia_{—} | 323325 | 4368216 |
| o_Bacteroidales_f_Bacteroidaceae_{—} | 4449055 | 365496 |
| g_Bacteroides_s_plebeius | | |

| | | | |
|---|---|---|---|
| p_Firmicutes_c_Clostridia_{—} | 367535 | 4464445 | 1667433 |
| o_Clostridiales_f_Lachnospiraceae_{—} | 4357353 | 196508 | 187338 |
| g_Dorea_s_{—} | 182416 | 293869 | 4008139 |
| | 189667 | 4242681 | 3673770 |
| | 4451907 | | |

| p_Actinobacteria_c_Coriobacteriia_{—} | 302647 | 303693 |
|---|---|---|
| o_Coriobacteriales_f_Coriobacteriaceae_{—} | 415315 | 189997 |
| g_Collinsella_s_{—} | | |
| p_Firmicutes_c_Bacilli_{—} | 301270 | |
| o_Lactobacillales_f_Streptococcaceae_{—} | 237444 | |
| g_Streptococcus_s_{—} | | |
| p_Firmicutes_c_Clostridia_{—} | 187470 | 176129 |
| o_Clostridiales_f_Lachnospiraceae_{—} | 177201 | 578511 |
| g_Coprococcus_s_{—} | | |

| p_Firmicutes_c_Erysipelotrichi_{—} | 4295707 | | |
|---|---|---|---|
| o_Erysipelotrichales_f_Erysipelotrichaceae_{—} | 179018 | | |
| g_[Eubacterium]_s_biforme | | | |
| p_Firmicutes_c_Clostridia_{—} | 133349 | | |
| o_Clostridiales_f_Clostridiaceae_{—} | | | |
| g_CandidatusArthromitus_s_{—} | | | |
| p_Firmicutes_c_Bacilli_{—} | 248902 | | |
| o_Turicibacterales_f_Turicibacteraceae_{—} | 347529 | | |
| g_Turicibacter_s_{—} | | | |
| p_Actinobacteria_c_Actinobacteria_{—} | 822770 | 69933 | 102049 |
| o_Bifidobacteriales_f_Bifidobacteriaceae_{—} | 825808 | 824876 | 471180 |
| g_Bifidobacterium_s_{—} | 4335781 | | |

*p = phylum, c = class, o = order, f = family, g = genus, s = species

TABLE 2

| Identification* | OTU Numbers | | | |
|---|---|---|---|---|
| p_Actinobacteria_c_Coriobacteriia_{—} | 4313430 | 231108 | | 230578 |
| o_Coriobacteriales_f_Coriobacteriaceae_{—} | 310028 | 293910 | | 4335376 |
| g_s_{—} | 188966 | 4397092 | | 4441081 |
| | 365033 | 302545 | | 305141 |
| | 366392 | 646800 | | |
| p_Firmicutes_c_Erysipelotrichi_{—} | 43628 | | | |
| o_Erysipelotrichales_f_Erysipelotrichaceae_{—} | 233573 | | | |
| g_[Eubacterium]_s_cylindroides | | | | |
| p_Actinobacteria_c_Actinobacteria_{—} | 370225 | | 359098 | |
| o_Bifidobacteriales_f_Bifidobacteriaceae_{—} | 235262 | | 4347159 | |
| g_Bifidobacterium_s_adolescentis | | | | |
| p_Firmicutes_c_Clostridia_{—} | 151623 | | 264967 | |
| o_Clostridiales_f_Veillonellaceae_{—} | 3039313 | | 52166 | |
| g_Megasphaera_s_{—} | 4452437 | | 266210 | |
| p_Firmicutes_c_Erysipelotrichi_{—} | 298651 | | 540924 | |
| o_Erysipelotrichales_f_Erysipelotrichaceae_{—} | 4312066 | | 337579 | |
| g_Bulleidia_s_{—} | 4302181 | | 274257 | |
| p_Actinobacteria_c_Actinobacteria_{—} | 72820 | | | |
| o_Bifidobacteriales_f_Bifidobacteriaceae_{—} | | | | |
| g_Bifidobacterium_s_longum | | | | |
| p_Actinobacteria_c_Coriobacteriia_{—} | 302647 | 414949 | | 415315 |
| o_Coriobacteriales_f_Coriobacteriaceae_{—} | 303693 | 290572 | | 147071 |
| g_Collinsella_s_{—} | 344601 | 189997 | | |
| p_Actinobacteria_c_Actinobacteria_{—} | 4481861 | | | |
| o_Bifidobacteriales_f_Bifidobacteriaceae_{—} | | | | |
| g_s_{—} | | | | |
| p_Actinobacteria_c_Coriobacteriia_{—} | 2990918 | | | |
| o_Coriobacteriales_f_Coriobacteriaceae_{—} | 288004 | | | |
| g_Collinsella_s_stercoris | 291811 | | | |
| p_Firmicutes_c_Clostridia_{—} | 4364564 | | | |
| o_Clostridiales_f_Lachnospiraceae_{—} | 335827 | | | |
| g_Butyrivibrio_s_{—} | | | | |
| p_Firmicutes_c_Erysipelotrichi_{—} | 147707 | | 297719 | |
| o_Erysipelotrichales_f_Erysipelotrichaceae_{—} | 195871 | | 323045 | |
| g_Bulleidia_s_p_1630_c5 | | | | |
| p_Firmicutes_c_Clostridia_{—} | 264552 | 4020046 | | 753638 |
| o_Clostridiales_f_Veillonellaceae_{—} | 1046997 | 174016 | | 403701 |
| g_Dialister_s_{—} | 4326870 | | | |
| p_Actinobacteria_c_Coriobacteriia_{—} | 4332878 | | 347783 | |
| o_Coriobacteriales_f_Coriobacteriaceae_{—} | 367068 | | 439547 | |
| g_Slackia_s_{—} | | | | |
| p_Bacteroidetes_c_Bacteroidia_{—} | 326482 | 558839 | | 4410166 |
| o_Bacteroidales_f_Prevotellaceae_{—} | 293843 | 568118 | | 307571 |
| g_Prevotella_s_copri | 321743 | 524891 | | 215670 |
| | 329693 | 527941 | | 4318208 |
| | 2075910 | 589329 | | 313121 |
| | 173565 | 4436552 | | 301253 |
| | 198786 | 346938 | | 196296 |
| | 184464 | 294270 | | 296442 |
| | 545061 | 328936 | | 292921 |
| | 925131 | 336372 | | 2280817 |
| | 292041 | 514512 | | 2037235 |
| | 509636 | 189083 | | 530653 |
| | 4412542 | 174831 | | 513003 |
| | 181539 | | | |
| p_Firmicutes_c_Erysipelotrichi_{—} | 293262 | | | |
| o_Erysipelotrichales_f_Erysipelotrichaceae_{—} | 4480861 | | | |
| g_Catenibacterium_s_{—} | 303221 | | | |
| p_Firmicutes_c_Clostridia_{—} | 287786 | | | |
| o_Clostridiales_f_Veillonellaceae_{—} | 2530636 | | | |
| g_Megamonas_s_{—} | 222842 | | | |
| p_Firmicutes_c_Bacilli_{—} | 178213 | | | |
| o_Lactobacillales_f_Lactobacillaceae_{—} | 4463108 | | | |
| g_Lactobacillus_s_ruminis | | | | |
| p_Firmicutes_c_Clostridia_{—} | 177228 | 268074 | | 328836 |
| o_Clostridiales_f_Clostridiaceae_{—} | 352846 | 327076 | | 4446320 |
| g_s_{—} | 309279 | 344578 | | 197329 |
| | 359750 | 196346 | | 1024529 |
| | 254446 | 308444 | | 178364 |
| | 195301 | 326637 | | 321096 |
| | 338956 | 261084 | | 1144996 |
| | 179536 | 290211 | | 188271 |
| | 315733 | 177423 | | 4387453 |
| | 355471 | 191803 | | 312935 |
| | 354258 | 270382 | | 306704 |
| | 327756 | 328955 | | 199268 |
| | 293594 | 4319938 | | 298514 |
| | 318091 | 297783 | | 291254 |
| | 341090 | 270200 | | 316228 |
| | 187466 | 294304 | | 325552 |
| | 182956 | 189503 | | 307302 |
| | 344553 | 1646171 | | 313142 |
| | 355269 | 193672 | | 182643 |
| | 4383953 | 2325032 | | 180516 |
| | 332764 | 341134 | | 298381 |
| | 356255 | 292489 | | 708285 |
| | 289679 | 314204 | | 350832 |
| | 180552 | 4468465 | | 322798 |
| | 305432 | 315529 | | 353784 |
| | 341054 | | | |
| p_Proteobacteria_c_Deltaproteobacteria_{—} | 30569 | | | |
| o_Desulfovibrionale_f_Desulfovibrionaceae_{—} | | | | |
| g_Desulfovibrio_s_{—} | | | | |
| p_Firmicutes_c_Clostridia_{—} | 4448928 | 215963 | | 310354 |
| o_Clostridiales_f_Clostridiaceae_{—} | 3438276 | 303990 | | 1846390 |
| g_Clostridium_s_{—} | 363389 | 292257 | | 3931537 |
| | 316267 | 323115 | | 4445673 |
| | 4401045 | 317533 | | 309658 |
| | 357529 | 306035 | | 292299 |
| | 302614 | 174516 | | 310954 |
| | 314402 | 311207 | | 306412 |
| p_Firmicutes_c_Bacilli_{—} | 292424 | 303161 | | 296659 |
| o_Lactobacillales_f_Streptococcaceae_{—} | 290735 | 288235 | | 299918 |
| g_Streptococcus_s_luteciae | 15458 | | | |
| p_Firmicutes_c_Clostridia_{—} | 290241 | 4370657 | | 4412788 |
| o_Clostridiales_f_Clostridiaceae_{—} | 304779 | 299207 | | 315982 |
| g_Clostridium_s_perfringens | 300501 | 289714 | | 4479317 |
| | 295411 | 302597 | | |
| p_Firmicutes_c_Clostridia_{—} | 3903651 | 316925 | | 180468 |
| o_Clostridiales_f_Ruminococcaceae_{—} | 548686 | 4420206 | | 308759 |
| g_Oscillospira_s_{—} | 175336 | 4357315 | | 589076 |
| | 585227 | 334215 | | 190676 |
| | 321484 | 1504042 | | 263546 |
| | 532922 | 4437359 | | 348009 |
| | 544996 | 106786 | | 317633 |
| | 839964 | | | |
| p_Firmicutes_c_Clostridia_{—} | 326430 | 351084 | | 347131 |
| o_Clostridiales_f_Clostridiaceae_{—} | 302610 | 197510 | | 311402 |
| g_Clostridium_s_hiranonis | 314749 | 309107 | | 1960569 |
| | 582379 | 290314 | | 4070491 |
| p_Firmicutes_c_Clostridia_{—} | 181167 | 187338 | | 4433417 |
| o_Clostridiales_f_Lachnospiraceae_{—} | 175978 | 4464445 | | 195081 |
| g_Dorea_s_{—} | 3185810 | 185603 | | 3150722 |
| | 4374302 | 4424111 | | 182653 |
| | 189396 | 230232 | | 305329 |
| | 3673770 | 181871 | | 38415 |
| | 176980 | 77458 | | 4451907 |
| | 182416 | 3205714 | | 197050 |
| | 193509 | 178616 | | 195999 |
| | 4436046 | 1667433 | | |
| p_Bacteroidetes_c_Bacteroidia_{—} | 323303 | 347875 | | 4307094 |
| o_Bacteroidales_f_[Paraprevotellaceae]_{—} | 4450194 | 1143551 | | 4449525 |
| g_[Prevotella]_s_{—} | 4474759 | 423264 | | 332968 |
| | 1136390 | 4385760 | | |
| p_Bacteroidetes_c_Bacteroidia_{—} | 4370491 | | 300859 | |
| o_Bacteroidales_f_Prevotellaceae_{—} | 4434579 | | 3754778 | |
| g_Prevotella_s_{—} | 4378740 | | 158423 | |
| p_Bacteroidetes_c_Bacteroidia_{—} | 4365130 | | | |
| o_Bacteroidales_f_Porphyromonadaceae_{—} | 585914 | | | |
| g_Parabacteroides_s_distasonis | 578016 | | | |
| p_Firmicutes_c_Clostridia_{—} | 183288 | 189459 | | 177359 |
| o_Clostridiales_f_Lachnospiraceae_{—} | 185667 | 175389 | | 187212 |
| g_Coprococcus_s_{—} | 325126 | 174019 | | 578437 |
| | 182289 | 181853 | | 195189 |
| | 191238 | 184013 | | 182903 |
| | 187470 | 578511 | | 2740950 |
| | 192218 | 175438 | | 177201 |
| | 187868 | 177760 | | 199077 |
| | 1678333 | 197603 | | 181560 |
| | 179911 | 188047 | | 184525 |
| | 2065341 | 177172 | | 187569 |
| | 181269 | 271449 | | 205613 |
| | 184656 | 183799 | | 178686 |
| p_Bacteroidetes_c_[Saprospirae]_{—} | 4422872 | | | |
| o_[Saprospirales]_f_Chitinophagaceae_{—} | 50765 | | | |
| g_Sediminibacterium_s_{—} | 808071 | | | |
| p_Proteobacteria_c_Betaproteobacteria_{—} | 1116384 | | 254888 | |
| o_Burkholderiales_f_Comamonadaceae_{—} | 1000148 | | 899348 | |
| g_s_{—} | | | | |
| p_Firmicutes_c_Clostridia_{—} | 294499 | 179512 | | 353392 |
| o_Clostridiales_f_Clostridiaceae_{—} | 347965 | 326083 | | 196315 |
| g_SMB53_s_{—} | 198209 | 289373 | | |
| p_Firmicutes_c_Clostridia_{—} | 178859 | 147969 | | 353632 |
| o_Clostridiales_f_Ruminococcaceae_{—} | 177800 | 179572 | | 291644 |
| g_Ruminococcus_s_{—} | 181035 | 259922 | | 4331723 |
| | 268720 | 2943548 | | 523140 |
| | 192598 | 323135 | | 341765 |
| | 405780 | 4456702 | | 223059 |
| | 146554 | 163243 | | 3235048 |
| | 4326091 | 177403 | | 207994 |
| | 2979308 | | | |

| | | | |
|---|---|---|---|
| p_Bacteroidetes_c_Bacteroidia_{—} | 198865 | 460953 | 180077 |
| o_Bacteroidales_f_S24_7_{—} | 196672 | 321735 | 38278 |
| g_s_{—} | 269726 | 197623 | 262148 |
| | 134762 | 175706 | 185550 |
| | 264352 | 187028 | 175598 |
| | 175646 | 235017 | 264734 |
| | 3231096 | 215495 | 263420 |
| | 209446 | 162639 | 176100 |
| | 216495 | 209030 | 259012 |
| | 198201 | 204003 | 196733 |
| | 271418 | 2212505 | 175458 |
| | 182945 | 342962 | 331720 |
| | 189778 | 185614 | 264657 |
| | 206817 | 192494 | 193038 |
| | 177115 | 209028 | 177512 |
| | 228730 | 275339 | 262166 |
| | 801260 | 324013 | 194830 |
| | 261350 | 177371 | 337004 |
| | 320169 | 2435303 | 173852 |
| | 174056 | 345330 | 302663 |
| | 174573 | 211820 | 178546 |
| | 174805 | 331772 | 430194 |
| | 181605 | 277364 | 420345 |
| | 258849 | 304088 | 178114 |
| | 190573 | 348038 | 185695 |
| | 330772 | 203713 | 178068 |
| | 3172943 | 174500 | |
| | 194043 | | |

| p_Proteobacteria_c_Deltaproteobacteria_{—} | 2897325 | |
|---|---|---|
| o_Desulfovibrionales_f_Desulfovibrionaceae_{—} | 359872 | |
| g_Bilophila_s_{—} | | |
| p_Bacteroidetes_c_Bacteroidia_{—} | 1726408 | 522582 |
| o_Bacteroidales_f_Porphyromonadaceae_{—} | 1952 | 4418496 |
| g_Parabacteroides_s_{—} | | |
| p_Firmicutes_c_Clostridia_{—} | 4424063 | 3779973 |
| o_Clostridiales_f_Lachnospiraceae_{—} | 360962 | 4232048 |
| g_Dorea_s_formicigenerans | | |

*p = phylum, c = class, o = order, f = family, g = genus, s = species

The present methods can be applicable to companion animals. In one aspect, the companion animal can be a feline. In one specific aspect, the feline can be at least 6 months old.

Another embodiment of the present invention includes a method of predicting percent of adult body fat for a companion animal having an age from 1 day to 6 months, comprising measuring the relative abundance of bacteria from a microbiome of the companion animal including Coprococcus spp, Candidatus Arthromitus spp, Turicibacter spp, [Eubacterium] biforme, Bifidobacterium spp, Streptococcus spp, Collinsella spp, Dorea spp, Clostridiales, Slackia spp, Erysipelotrichaceae, Faecalibacterium prausnitzii, Bacteroides spp, Ruminococcus spp, Phascolarctobacterium spp, Bacteroides plebeius; and calculating the percent of adult body fat according to the equation:

$$Predicted adult body fat \% = ( about ( - 30 ) \times ( relative abundance of Coprococcus spp ) ) + \\ ( about ( - 18.5 ) \times ( relative abundance of CandidatusArthromitus spp ) ) + \\ ( about ( - 1.5 ) \times ( relative abundance of Turicibacter spp ) ) + ( about ( - 0.1 ) \times ( relative abundance of [ Eubacterium ] biforme ) ) + ( about ( - 0.19 ) \times ( relative abundance of Bifidobacterium spp ) ) + ( about ( - 0.05 ) \times ( relative abundance of Streptococcus spp ) ) + ( about ( 0.10 ) \times ( relative abundance of Collinsella spp ) ) + ( about ( 0.4 ) \times ( relative abundance of Dorea spp ) ) + ( about ( 0.6 ) \times ( relative abundance of Clostridiales ) ) + ( about ( 3.4 ) \times ( relative abundance of Slackia spp ) ) + ( about ( 9 ) \times ( relative abundance of Erysipelotrichceae ) ) + ( about ( 11 ) \times ( relative abundance of Faecalibacterium prausnitzii ) ) + ( about ( 21 ) \times ( relative abundance of Bacteroides spp ) ) + ( about ( 24 ) \times ( relative abundance of Ruminococcus spp ) ) + ( about ( 26 ) \times ( relative abundance of Phascolarctobacterium spp ) ) + ( about ( 69 ) \times ( relative abundance of Bacteroides plebeius ) ) .$$

In one embodiment, the companion animal can be a feline. In one aspect, the term “about” provides a 5% range for each numerical or calculated value. In specific aspects, the term “about” provides a 2% range, or even a 1% range for each numerical or calculated value.

In another embodiment, the equation can be:

$$Predicted adult body fat \% = ( ( - 30.7521 ) \times ( relative abundance of Coprococcus spp ) ) + \\ ( ( - 18.6353 ) \times ( relative abundance of CandidatusArthromitus spp ) ) + \\ ( ( - 1.61918 ) \times ( relative abundance of Turicibacter spp ) ) + ( ( - 0.10591 ) \times ( relative abundance of [ Eubacterium ] biforme ) ) + ( ( - 0.09779 ) \times ( relative abundance of Bifidobacterium spp ) ) + ( ( - 0.050793 ) \times ( relative abundance of Streptococcus spp ) ) + ( ( 0.096472 ) \times ( relative abundance of Collinsella spp ) ) + ( ( 0.413818 ) \times ( relative abundance of Dorea spp ) ) + ( ( 0.6271 ) \times ( relative abundance of Clostridiales ) ) + ( ( 3.37069 ) \times ( relative abundance of Slackia spp ) ) + ( ( 8.97799 ) \times ( relative abundance of Erysipelotrichceae ) ) + ( ( 11.0669 ) \times ( relative abundance of Faecalibacterium prausnitzii ) ) + ( ( 21.1541 ) \times ( relative abundance of Bacteroides spp ) ) + ( ( 24.0743 ) \times ( relative abundance of Ruminococcus spp ) ) + ( ( 25.8582 ) \times ( relative abundance of Phascolarctobacterium spp ) ) + ( ( 69.3693 ) \times ( relative abundance of Bacteroides plebeius ) ) .$$

### EXAMPLES

The invention can be further illustrated by the following example, although it will be understood that this example is included merely for purposes of illustration and is not intended to limit the scope of the invention unless otherwise specifically indicated.

### Example 1

### Kitten Study

Fecal samples were obtained from 31 weanling kittens (8 to 14 weeks of age). Fecal microbiome was determined using 454 pyrosequencing of 16S rRNA genes. Kittens were fed a dry cat food until 9 months of age. At that time, body fat was determined by DEXA (Dual-energy X-ray absorptiometry). Fecal microbiome (relative abundance of bacteria) of the weanling kittens was used to predict body fat at 9 months of age according to the correlations in Table 3 and the following equation.

TABLE 3

| | | correlated |
|---|---|---|
| Identification* | p(corr) | with |
| p_Bacteroidetes_c_Bacteroidia_{—} | 0.520634 | over- |
| o_Bacteroidales_f_Bacteroidaceae_{—} | | weight/ |
| g_Bacteroides_s_{—} | | higher |
| | | body fat |
| **p_Firmicutes_c_Clostridia_{—} | 0.436181 | over- |
| o_Clostridiales_f_Veillonellaceae_{—} | | weight/ |
| g_Phascolarctobacterium_s_{—} | | higher |
| | | body fat |
| **p_Firmicutes_c_Clostridia_{—} | 0.432632 | over- |
| o_Clostridiales_f_Ruminococcaceae_{—} | | weight/ |
| g_Faecalibacterium_s_prausnitzii | | higher |
| | | body fat |
| **p_Firmicutes_c_Erysipelotrichi_{—} | 0.428768 | over- |
| o_Erysipelotrichales_f_Erysipelotrichaceae_{—} | | weight/ |
| g_s_{—} | | higher |
| | | body fat |
| p_Actinobacteria_c_Coriobacteriia_{—} | 0.419778 | over- |
| o_Coriobacteriales_f_Coriobacteriaceae_{—} | | weight/ |
| g_Slackia_s_{—} | | higher |
| | | body fat |
| **p_Firmicutes_c_Clostridia_{—} | 0.404307 | over- |
| o_Clostridiales_f_g_s_{—} | | weight/ |
| | | higher |
| | | body fat |
| **p_Firmicutes_c_Clostridia_{—} | 0.397102 | over- |
| o_Clostridiales_f_Ruminococcaceae_{—} | | weight/ |
| g_Ruminococcus_s_{—} | | higher |
| | | body fat |
| p_Bacteroidetes_c_Bacteroidia_{—} | 0.390707 | over- |
| o_Bacteroidales_f_Bacteroidaceae_{—} | | weight/ |
| g_Bacteroides_s_plebeius | | higher |
| | | body fat |
| **p_Firmicutes_c_Clostridia_{—} | 0.379123 | over- |
| o_Clostridiales_f_Lachnospiraceae_{—} | | weight/ |
| g_Dorea_s_{—} | | higher |
| | | body fat |
| p_Actinobacteria_c_Coriobacteriia_{—} | 0.142404 | over- |
| o_Coriobacteriales_f_Coriobacteriaceae_{—} | | weight/ |
| g_Collinsella_s_{—} | | higher |
| | | body fat |
| **p_Firmicutes_c_Bacilli_{—} | 0.0981911 | over- |
| o_Lactobacillales_f_Streptococcaceae_{—} | | weight/ |
| g_Streptococcus_s_{—} | | higher |
| | | body fat |
| **p_Firmicutes_c_Clostridia_{—} | −0.348361 | thin/lower |
| o_Clostridiales_f_Lachnospiraceae_{—} | | body fat |
| g_Coprococcus_s_{—} | | |
| **p_Firmicutes_c_Erysipelotrichi_{—} | −0.374887 | thin/lower |
| o_Erysipelotrichales_f_Erysipelotrichaceae_{—} | | body fat |
| g_[Eubacterium]_s_biforme | | |
| **p_Firmicutes_c_Clostridia_{—} | −0.410485 | thin/lower |
| o_Clostridiales_f_Clostridiaceae_{—} | | body fat |
| g_CandidatusArthromitus_s_{—} | | |
| **p_Firmicutes_c_Bacilli_{—} | −0.411504 | thin/lower |
| o_Turicibacterales_f_Turicibacteraceae_{—} | | body fat |
| g_Turicibacter_s_{—} | | |
| p_Actinobacteria_c_Actinobacteria_{—} | −0.617376 | thin/lower |
| o_Bifidobacteriales_f_Bifidobacteriaceae_{—} | | body fat |
| g_Bifidobacterium_s_{—} | | |

*p = phylum, c = class, o = order, f = family, g = genus, s = species
**= Known firmicutes correlated with overweight in humans

$$Predicted adult body fat \% = ( ( - 30.7521 ) \times ( relative abundance of Coprococcus spp ) ) + \\ ( ( - 18.6353 ) \times ( relative abundance of CandidatusArthromitus spp ) ) + \\ ( ( - 1.61918 ) \times ( relative abundance of Turicibacter spp ) ) + ( ( - 0.10591 ) \times ( relative abundance of [ Eubacterium ] biforme ) ) + ( ( - 0.09779 ) \times ( relative abundance of Bifidobacterium spp ) ) + ( ( - 0.050793 ) \times ( relative abundance of Streptococcus spp ) ) + ( ( 0.096472 ) \times ( relative abundance of Collinsella spp ) ) + ( ( 0.413818 ) \times ( relative abundance of Dorea spp ) ) + ( ( 0.6271 ) \times ( relative abundance of Clostridiales ) ) + ( ( 3.37069 ) \times ( relative abundance of Slackia spp ) ) + ( ( 8.97799 ) \times ( relative abundance of Erysipelotrichceae ) ) + ( ( 11.0669 ) \times ( relative abundance of Faecalibacterium prausnitzii ) ) + ( ( 21.1541 ) \times ( relative abundance of Bacteroides spp ) ) + ( ( 24.0743 ) \times ( relative abundance of Ruminococcus spp ) ) + ( ( 25.8582 ) \times ( relative abundance of Phascolarctobacterium spp ) ) + ( ( 69.3693 ) \times ( relative abundance of Bacteroides plebeius ) ) .$$

As noted in Table 3, various firmicutes that are typically correlated with being overweight in humans and other species (e.g., rodents) were presently found as predicting development of being overweight and predicting remaining lean.

### Example 2

### Adult Cat Study

Fecal samples were obtained from 15 thin and 14 overweight cats. Fecal microbiome was determined using 454 pyrosequencing of 16S rRNA genes. Fecal microbiome (relative abundance of bacteria) of the cats was correlated with body condition (thin or overweight) according to Table 4.

TABLE 4

| | | correlated |
|---|---|---|
| Identification* | p(corr) | with |
| p_Actinobacteria_c_Coriobacteriia_{—} | 0.647438 | over- |
| o_Coriobacteriales_f_Coriobacteriaceae_{—} | | weight |
| g_s_{—} | | |
| p_Firmicutes_c_Erysipelotrichi_{—} | 0.541646 | over- |
| o_Erysipelotrichales_f_Erysipelotrichaceae_{—} | | weight |
| g_[Eubacterium]_s_cylindroides | | |
| p_Actinobacteria_c_Actinobacteria_{—} | 0.537301 | over- |
| o_Bifidobacteriales_f_Bifidobacteriaceae_{—} | | weight |
| g_Bifidobacterium_s_adolescentis | | |
| p_Firmicutes_c_Clostridia_{—} | 0.51891 | over- |
| o_Clostridiales_f_Veillonellaceae_{—} | | weight |
| g_Megasphaera_s_{—} | | |
| p_Firmicutes_c_Erysipelotrichi_{—} | 0.453303 | over- |
| o_Erysipelotrichales_f_Erysipelotrichaceae_{—} | | weight |
| g_Bulleidia_s_{—} | | |
| p_Actinobacteria_c_Actinobacteria_{—} | 0.421699 | over- |
| o_Bifidobacteriales_f_Bifidobacteriaceae_{—} | | weight |
| g_Bifidobacterium_s_longum | | |
| p_Actinobacteria_c_Coriobacteriia_{—} | 0.396894 | over- |
| o_Coriobacteriales_f_Coriobacteriaceae_{—} | | weight |
| g_Collinsella_s_{—} | | |
| p_Actinobacteria_c_Actinobacteria_{—} | 0.382441 | over- |
| o_Bifidobacteriales_f_Bifidobacteriaceae_{—} | | weight |
| g_s | | |
| p_Actinobacteria_c_Coriobacteriia_{—} | 0.365941 | over- |
| o_Coriobacteriales_f_Coriobacteriaceae_{—} | | weight |
| g_Collinsella_s_stercoris | | |
| p_Firmicutes_c_Clostridia_{—} | 0.357648 | over- |
| o_Clostridiales_f_Lachnospiraceae_{—} | | weight |
| g_Butyrivibrio_s_{—} | | |
| p_Firmicutes_c_Erysipelotrichi_{—} | 0.328821 | over- |
| o_Erysipelotrichales_f_Erysipelotrichaceae_{—} | | weight |
| g_Bulleidia_s_p_1630_c5 | | |
| p_Firmicutes_c_Clostridia_{—} | 0.314879 | over- |
| o_Clostridiales_f_Veillonellaceae_{—} | | weight |
| g_Dialister_s_{—} | | |
| p_Actinobacteria_c_Coriobacteriia_{—} | 0.308146 | over- |
| o_Coriobacteriales_f_Coriobacteriaceae_{—} | | weight |
| g_Slackia_s_{—} | | |
| p_Bacteroidetes_c_Bacteroidia_{—} | 0.296077 | over- |
| o_Bacteroidales_f_Prevotellaceae_{—} | | weight |
| g_Prevotella_s_copri | | |
| p_Firmicutes_c_Erysipelotrichi_{—} | 0.293355 | over- |
| o_Erysipelotrichales_f_Erysipelotrichaceae_{—} | | weight |
| g_Catenibacterium_s_{—} | | |
| p_Firmicutes_c_Clostridia_{—} | 0.284066 | over- |
| o_Clostridiales_f_Veillonellaceae_{—} | | weight |
| g_Megamonas_s_{—} | | |
| p_Firmicutes_c_Bacilli_{—} | 0.212153 | over- |
| o_Lactobacillales_f_Lactobacillaceae_{—} | | weight |
| g_Lactobacillus_s_ruminis | | |
| p_Firmicutes_c_Clostridia_{—} | −0.19087 | thin |
| o_Clostridiales_f_Clostridiaceae_{—} | | |
| g_s_{—} | | |
| p_Proteobacteria_c_Deltaproteobacteria_{—} | −0.21596 | thin |
| o_Desulfovibrionale_f_Desulfovibrionaceae_{—} | | |
| g_Desulfovibrio_s_{—} | | |
| p_Firmicutes_c_Clostridia_{—} | −0.23624 | thin |
| o_Clostridiales_f_Clostridiaceae_{—} | | |
| g_Clostridium_s_{—} | | |
| p_Firmicutes_c_Bacilli_{—} | −0.24144 | thin |
| o_Lactobacillales_f_Streptococcaceae_{—} | | |
| g_Streptococcus_s_luteciae | | |
| p_Firmicutes_c_Clostridia_{—} | −0.25102 | thin |
| o_Clostridiales_f_Clostridiaceae_{—} | | |
| g_Clostridium_s_perfringens | | |
| p_Firmicutes_c_Clostridia_{—} | −0.25137 | thin |
| o_Clostridiales_f_Ruminococcaceae_{—} | | |
| g_Oscillospira_s_{—} | | |
| p_Firmicutes_c_Clostridia_{—} | −0.25797 | thin |
| o_Clostridiales_f_Clostridiaceae_{—} | | |
| g_Clostridium_s_hiranonis | | |
| p_Firmicutes_c_Clostridia_{—} | −0.26763 | thin |
| o_Clostridiales_f_Lachnospiraceae_{—} | | |
| g_Dorea_s_{—} | | |
| p_Bacteroidetes_c_Bacteroidia_{—} | −0.27187 | thin |
| o_Bacteroidales_f_[Paraprevotellaceae]_{—} | | |
| g_[Prevotella]_s_{—} | | |
| p_Bacteroidetes_c_Bacteroidia_{—} | −0.31754 | thin |
| o_Bacteroidales_f_Prevotellaceae_{—} | | |
| g_Prevotella_s_{—} | | |
| p_Bacteroidetes_c_Bacteroidia_{—} | −0.32447 | thin |
| o_Bacteroidales_f_Porphyromonadaceae_{—} | | |
| g_Parabacteroides_s_distasonis | | |
| p_Firmicutes_c_Clostridia_{—} | −0.33226 | thin |
| o_Clostridiales_f_Lachnospiraceae_{—} | | |
| g_Coprococcus_s_{—} | | |
| p_Bacteroidetes_c_[Saprospirae]_{—} | −0.33405 | thin |
| o_[Saprospirales]_f_Chitinophagaceae_{—} | | |
| g_Sediminibacteriurn_s_{—} | | |
| p_Proteobacteria_c_Betaproteobacteria_{—} | −0.3356 | thin |
| o_Burkholderiales_f_Comamonadaceae_{—} | | |
| g_s_{—} | | |
| p_Firmicutes_c_Clostridia_{—} | −0.34111 | thin |
| o_Clostridiales_f_Clostridiaceae_{—} | | |
| g_SMB53_s_{—} | | |
| p_Firmicutes_c_Clostridia_{—} | −0.37086 | thin |
| o_Clostridiales_f_Ruminococcaceae_{—} | | |
| g_Ruminococcus_s_{—} | | |
| p_Bacteroidetes_c_Bacteroidia_{—} | −0.38788 | thin |
| o_Bacteroidales_f_S24_7_{—} | | |
| g_s_{—} | | |
| p_Proteobacteria_c_Deltaproteobacteria_{—} | −0.39987 | thin |
| o_Desulfovibrionales_f_Desulfovibrionaceae_{—} | | |
| g_Bilophila_s_{—} | | |
| p_Bacteroidetes_c_Bacteroidia_{—} | −0.40802 | thin |
| o_Bacteroidales_f_Porphyromonadaceae_{—} | | |
| g_Parabacteroides_s_{—} | | |
| p_Firmicutes_c_Clostridia_{—} | −0.44036 | thin |
| o_Clostridiales_f_Lachnospiraceae_{—} | | |
| g_Dorea_s_formicigenerans | | |

*p = phylum, c = class, o = order, f = family, g = genus, s = species

In the specification, there have been disclosed typical embodiments of the invention. Although specific terms are employed, they are used in a generic and descriptive sense only and not for purposes of limitation. The scope of the invention is set forth in the claims. Obviously many modifications and variations of the invention are possible in light of the above teachings. It is therefore to be understood that within the scope of the appended claims the invention may be practiced otherwise than as specifically described.

What is claimed is:

1. A method for determining overweight risk in a companion animal, comprising:
measuring a relative abundance of bacteria from a microbiome of the companion animal including at least two bacterium selected from the group consisting of Bifidobacterium longum, Coriobacteriaceae, [Eubacterium] cylindroides, Bifidobacterium adolescentis, Megasphaera, Bulleidia, Collinsella spp, Bifidobacteriumceae, Collinsella stercoris, Butyrivibrio, Bulleidia p_1630_c5, Dialister, Slackia spp, Prevotella copri, Catenibacterium, Megamonas, Lactobacillus ruminis, Clostridiaceae, Desulfovibrio, Clostridium, Streptococcus luteciae, Clostridium perfringens, Oscillospira, Clostridium hiranonis, Dorea spp, [Paraprevotellaceae] [Prevotella], Prevotella, Parabacteroides distasonis, Coprococcus spp, Sediminibacterium, Comamonadaceae, SMB53, Ruminococcus spp, S24_7_g, Bilophila, Parabacteroides, and Dorea formicigenerans;
comparing the relative abundance of the bacteria to a relative abundance of the bacteria in a lean microbiome profile or in an overweight microbiome profile; and
determining that the companion animal is at risk for being overweight if the relative abundance of bacteria is within the overweight microbiome profile or if the relative abundance of bacteria is outside the lean microbiome profile.

2. The method of claim 1, wherein the determining step is based on comparing to the lean microbiome profile.

3. The method of claim 1, wherein the lean microbiome profile includes at least two bacterium selected from the group consisting of: Clostridiaceae, Desulfovibrio, Clostridium, Streptococcus luteciae, Clostridium perfringens, Oscillospira, Clostridium hiranonis, Dorea spp, [Paraprevotellaceae] [Prevotella], Prevotella, Parabacteroides distasonis, Coprococcus spp, Sediminibacterium, Comamonadaceae, SMB53, Ruminococcus spp, S24_7_g, Bilophila, Parabacteroides, and Dorea formicigenerans.

4. The method of claim 3, wherein the relative abundance of Clostridiaceae in the lean microbiome profile ranges from 0.07% to 6.7%, the relative abundance of Desulfovibrio in the lean microbiome profile ranges from 0.001% to 0.75%, the relative abundance of Clostridium in the lean microbiome profile ranges from 0.001% to 7.7%, the relative abundance of Streptococcus luteciae in the lean microbiome profile ranges from 0.001% to 3%, the relative abundance of Clostridium perfringens in the lean microbiome profile ranges from 0.001% to 1.1%, the relative abundance of Oscillospira in the lean microbiome profile ranges from 0.02% to 0.77%, the relative abundance of Clostridium hiranonis in the lean microbiome profile ranges from 0.9% to 17%, the relative abundance of Dorea spp in the lean microbiome profile ranges from 0.001% to 1%, the relative abundance of [Paraprevotellaceae] [Prevotella] in the lean microbiome profile ranges from 0.001% to 6.5%, the relative abundance of Prevotella in the lean microbiome profile ranges from 0.001% to 0.6%, the relative abundance of Parabacteroides distasonis in the lean microbiome profile ranges from 0.001 to 0.4%, the relative abundance of Coprococcus spp in the lean microbiome profile ranges from 0.001% to 1.6%, the relative abundance of Sediminibacterium in the lean microbiome profile ranges from 0.001% to 0.15%, the relative abundance of Comamonadaceae in the lean microbiome profile ranges from 0.001% to 0.31%, the relative abundance of SMB53 in the lean microbiome profile ranges from 0.03% to 0.8%, the relative abundance of Ruminococcus spp in the lean microbiome profile ranges from 0.001% to 1.6%, the relative abundance of S24_7_g in the lean microbiome profile ranges from 0.001% to 23%, the relative abundance of Bilophila in the lean microbiome profile ranges from 0.001% to 0.1%, the relative abundance of Parabacteroides in the lean microbiome profile ranges from 0.001% to 1.4%, and the relative abundance of Dorea formicigenerans in the lean microbiome profile ranges from 0.001% to 0.65%.

5. The method of claim 1, wherein the determining step is based on comparing to the overweight microbiome profile.

6. The method of claim 1, wherein the overweight microbiome profile includes at least two bacterium selected from the group consisting of: Bifidobacterium longum, Coriobacteriaceae, [Eubacterium] cylindroides, Bifidobacterium adolescentis, Megasphaera, Bulleidia, Collinsella spp, Bifidobacteriumceae, Collinsella stercoris, Butyrivibrio, Bulleidia p_1630_c5, Dialister, Slackia spp, Prevotella copri, Catenibacterium, Megamonas, and Lactobacillus ruminis.

7. The method of claim 6, wherein the relative abundance of Bifidobacterium longum in the overweight microbiome profile ranges from 0.001% to 1.61%, the relative abundance of Coriobacteriaceae in the overweight microbiome profile ranges from 0.001% to 24.1%, the relative abundance of [Eubacterium] cylindroides in the overweight microbiome profile ranges from 0.06% to 1%, the relative abundance of Bifidobacterium adolescentis in the overweight microbiome profile ranges from 0.001% to 17.3%, the relative abundance of Megasphaera in the overweight microbiome profile ranges from 0.001% to 12.5%, the relative abundance of Bulleidia in the overweight microbiome profile ranges from 0.001% to 3.4%, the relative abundance of Collinsella spp in the overweight microbiome profile ranges from 0.44% to 6.5%, the relative abundance of Bifidobacteriumceae in the overweight microbiome profile ranges from 0.065% to 0.95%, the relative abundance of Collinsella stercorin in the overweight microbiome profile ranges from 0.28% to 2%, the relative abundance of Butyrivibrio in the overweight microbiome profile ranges from 0.001% to 0.14%, the relative abundance of Bulleidia p_1630_c5 in the overweight microbiome profile ranges from 0.4 to 1.9%, the relative abundance of Dialister in the overweight microbiome profile ranges from 0.001% to 5.9%, the relative abundance of Slackia spp in the overweight microbiome profile ranges from 0.01% to 0.32%, the relative abundance of Prevotella copri in the overweight microbiome profile ranges from 2% to 18%, the relative abundance of Catenibacterium in the overweight microbiome profile ranges from 0.001% to 3.5%, the relative abundance of Megamonas in the overweight microbiome profile ranges from 0.001% to 0.19%, and the relative abundance of Lactobacillus ruminis in the overweight microbiome profile ranges from 0.001% to 4.3%.

8. The method of claim 1, wherein the bacteria are from different genuses.

9. The method of claim 1, wherein the bacteria are from different families.

10. The method of claim 1, wherein the bacteria are from different orders.

11. The method of claim 1, wherein the bacteria are from different classes.

12. The method of claim 1, wherein the bacteria are from different phyla.

13. The method of claim 1, wherein the bacteria include at least 3 bacterium.

14. The method of claim 1, wherein the bacteria include at least 4 bacterium.

15. The method of claim 1, wherein the bacteria include Megasphaera, Bifidobacterium, and Prevotella copri.

16. The method of claim 1, wherein the companion animal is a feline having an age of at least 6 months.

17. A method of predicting percent of adult body fat for a companion animal having an age from 1 day to 6 months, comprising
measuring the relative abundance of bacteria from a microbiome of the companion animal including
Coprococcus spp, Candidatus Arthromitus spp, Turicibacter spp, [Eubacterium] biforme, Bifidobacterium spp, Streptococcus spp, Collinsella spp, Dorea spp, Clostridiales, Slackia spp, Erysipelotrichaceae, Faecalibacterium prausnitzii, Bacteroides spp, Ruminococcus spp, Phascolarctobacterium spp, Bacteroides plebeius; and
calculating the percent of adult body fat according to the equation:
$$Predicted adult body fat \% = ( about ( - 30 ) \times ( relative abundance of Coprococcus spp ) ) + \\ ( about ( - 18.5 ) \times ( relative abundance of CandidatusArthromitus spp ) ) + \\ ( about ( - 1.5 ) \times ( relative abundance of Turicibacter spp ) ) + ( about ( - 0.1 ) \times ( relative abundance of [ Eubacterium ] biforme ) ) + ( about ( - 0.19 ) \times ( relative abundance of Bifidobacterium spp ) ) + ( about ( - 0.05 ) \times ( relative abundance of Streptococcus spp ) ) + ( about ( 0.10 ) \times ( relative abundance of Collinsella spp ) ) + ( about ( 0.4 ) \times ( relative abundance of Dorea spp ) ) + ( about ( 0.6 ) \times ( relative abundance of Clostridiales ) ) + ( about ( 3.4 ) \times ( relative abundance of Slackia spp ) ) + ( about ( 9 ) \times ( relative abundance of Erysipelotrichceae ) ) + ( about ( 11 ) \times ( relative abundance of Faecalibacterium prausnitzii ) ) + ( about ( 21 ) \times ( relative abundance of Bacteroides spp ) ) + ( about ( 24 ) \times ( relative abundance of Ruminococcus spp ) ) + ( about ( 26 ) \times ( relative abundance of Phascolarctobacterium spp ) ) + ( about ( 69 ) \times ( relative abundance of Bacteroides plebeius ) ) .$$

18. The method of claim 17, where the equation is:
$$Predicted adult body fat \% = ( ( - 30.7521 ) \times ( relative abundance of Coprococcus spp ) ) + \\ ( ( - 18.6353 ) \times ( relative abundance of CandidatusArthromitus spp ) ) + \\ ( ( - 1.61918 ) \times ( relative abundance of Turicibacter spp ) ) + ( ( - 0.10591 ) \times ( relative abundance of [ Eubacterium ] biforme ) ) + ( ( - 0.09779 ) \times ( relative abundance of Bifidobacterium spp ) ) + ( ( - 0.050793 ) \times ( relative abundance of Streptococcus spp ) ) + ( ( 0.096472 ) \times ( relative abundance of Collinsella spp ) ) + ( ( 0.413818 ) \times ( relative abundance of Dorea spp ) ) + ( ( 0.6271 ) \times ( relative abundance of Clostridiales ) ) + ( ( 3.37069 ) \times ( relative abundance of Slackia spp ) ) + ( ( 8.97799 ) \times ( relative abundance of Erysipelotrichceae ) ) + ( ( 11.0669 ) \times ( relative abundance of Faecalibacterium prausnitzii ) ) + ( ( 21.1541 ) \times ( relative abundance of Bacteroides spp ) ) + ( ( 24.0743 ) \times ( relative abundance of Ruminococcus spp ) ) + ( ( 25.8582 ) \times ( relative abundance of Phascolarctobacterium spp ) ) + ( ( 69.3693 ) \times ( relative abundance of Bacteroides plebeius ) ) .$$

19. The method of claim 17, wherein the companion animal is a kitten.
